(19) United States
(12) Patent Application Publication
(10) Pub. No.: US 2025/0140394 A1
(43) Pub. Date: May 1, 2025

(54) METHOD FOR ACQUIRING USE INFORMATION OF MEDICAL IMAGING DEVICE, APPARATUS, AND SYSTEM

(71) Applicant: GE Precision Healthcare LLC, Waukesha, WI (US)

(72) Inventors: Jianchen Lu, Shanghai (CN); Da Luan, Shanghai (CN); Qingtao Shen, Shanghai (CN)

(21) Appl. No.: 18/927,699

(22) Filed: Oct. 25, 2024

(30) Foreign Application Priority Data
Oct. 27, 2023 (CN) 202311408861.9

(51) Int. Cl. G16H 40/60; G06F 16/31; G06F 16/383; G06V 30/146; G16H 30/20; G16H 40/40

(52) U.S. Cl. CPC G16H 40/60; G06F 16/31; G06F 16/383; G06V 30/147; G16H 30/20; G16H 40/40

(57) ABSTRACT

Provided in embodiments of the present application are a method for acquiring use information of a medical imaging device, an apparatus, and a system. The method for acquiring use information of a medical imaging device includes: acquiring an image sequence, wherein the image sequence is generated by the medical imaging device and includes a plurality of images in a time dimension; performing text recognition on a specific region of the images and generating text information; and generating use information of the medical imaging device according to the text information.

### TECHNICAL FIELD

Embodiments of the present application relate to the technical field of medical devices, and particularly relate to a method for acquiring use information of a medical imaging device, an apparatus, and a system.

### BACKGROUND

Medical institutions typically use a large variety of different medical imaging devices, such as ultrasound imaging devices, computed tomography (CT) devices, magnetic resonance imaging (MRI) devices, positron emission computed tomography (PET) devices, and so on. Various medical imaging devices may include different models of medical imaging devices provided by different device manufacturers. There is a need for medical institutions to manage this large variety of medical imaging devices, such as understanding the use conditions of the devices, performing predictive maintenance, improving the efficiency of use. For the purpose of managing these medical imaging devices, relevant information may be collected from the medical imaging devices.

It should be noted that the above introduction of the background is only set forth to help clearly and completely describe the technical solutions of the present application, and to facilitate the understanding of those skilled in the art.

### SUMMARY

The inventors have found that, for medical imaging devices, the information formats of different medical systems are different, and thus the workload of determining use information of each medical imaging device based on different medical systems is very large, and the efficiency of acquiring the use information of each medical imaging device is low.

To address at least one of the above technical problems, the embodiments of the present invention provide a method for acquiring use information of a medical imaging device, an apparatus, and a system.

According to one aspect of the embodiments of the present application, a method for acquiring use information of a medical imaging device is provided, the method comprising: acquiring an image sequence, wherein the image sequence is generated by the medical imaging device and comprises a plurality of images in a time dimension; performing text recognition on a specific region of the images and generating text information; and generating use information of the medical imaging device according to the text information.

According to one aspect of the embodiments of the present application, an imaging device management apparatus is provided, wherein the apparatus comprises: an acquisition unit configured to acquire an image sequence, wherein the image sequence is generated by a medical imaging device and comprises a plurality of images in a time dimension; a recognition unit configured to perform text recognition on a specific region of the images and generate text information; and a generation unit configured to generate use information of the medical imaging device according to the text information.

According to one aspect of the embodiments of the present application, a medical imaging system is provided, comprising: a medical imaging device configured to generate an image sequence, wherein the image sequence comprises a plurality of images in a time dimension; and a device management apparatus configured to acquire the image sequence, perform text recognition on a specific region of the images of the image sequence and generate text information, and generate use information of the medical imaging device according to the text information.

One of the beneficial effects of the embodiments of the present application is that: text recognition is performed on a specific region of an image in an image sequence generated by a medical imaging device, and use information of the medical imaging device is generated according to text information obtained by the recognition, such that the workload of text recognition can be reduced, computational resources can be saved, and the efficiency of generating the use information of the medical imaging device can be improved.

With reference to the following description and drawings, specific implementations of the embodiments of the present application are disclosed in detail, and the means by which the principles of the embodiments of the present application can be employed are illustrated. It should be understood that the implementations of the present application are not limited in scope thereby. Within the scope of the spirit and clauses of the appended claims, the implementations of the present application include many changes, modifications, and equivalents.

### BRIEF DESCRIPTION OF THE DRAWINGS

The included drawings are used to provide further understanding of the embodiments of the present application, which constitute a part of the description, and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive effort. In the drawings:

FIG. 1 is a schematic diagram of a method for acquiring use information of a medical imaging device according to an embodiment of the present application;

FIG. 2 is a schematic diagram of the implementation of step 104 according to an embodiment of the present application;

FIG. 3 is a schematic diagram of a specific region according to an embodiment of the present application;

FIG. 4 is a schematic diagram of an imaging device management apparatus according to an embodiment of the present application;

FIG. 5 is another schematic diagram of the imaging device management apparatus according to an embodiment of the present application;

FIG. 6 is a schematic diagram of a first computer device according to the present application; and

FIG. 7 is a schematic diagram of a medical imaging system according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and some of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of the described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations. The term “include/comprise” when used herein refers to the presence of features, integrated components, steps, or assemblies, but does not preclude the presence or addition of one or more other features, integrated components, steps, or assemblies.

The medical imaging device described herein may be suitable for a variety of medical imaging modalities, including but not limited to ultrasound imaging devices, endoscope devices, computed tomography (CT) devices, magnetic resonance imaging (MRI) devices, positron emission tomography (PET) devices, single photon emission computed tomography (SPECT) devices, PET/CT, PET/MR, or any other appropriate medical imaging devices.

Ultrasound imaging uses ultrasound beams for scanning to obtain image data by receiving and processing reflected signals.

Endoscopic imaging is as follows: a detection region is irradiated by a light source disposed at the front end of a probe, or by light rays from a rear light source after being transmitted to the front end through an optical fiber bundle, an object lens images the detection region on a photosensitive surface of a CCD, and optical signals are converted into electrical signals to obtain image data.

CT uses X-rays to perform continuous cross-sectional scanning around a certain part of a scan subject, and the X-rays that pass through the section are received by a detector and transformed into visible light, or a received photon signal is directly converted to form medical image data after a series of processing.

MRI forms an image by means of reconstruction, based on the principle of nuclear magnetic resonance of atomic nuclei, by transmitting radio frequency pulses to the scan subject and receiving electromagnetic signals released by the scan subject.

PET uses a cyclotron to accelerate charged particles to bombard a target nucleus, which produces positron-bearing radionuclides by means of nuclear reactions and synthesizes imaging agents that are introduced into the body and localized in a target organ. The radionuclides emit positively charged electrons during a decay process, and after a positron travels a short distance in the tissue, the positron interacts with the electrons in the surrounding material and annihilation radiation occurs, from which two photons of equal energy are emitted in opposite directions. PET imaging uses a series of paired detectors that are arranged 180 degrees from each other and that receive coincidence lines to detect the photons of annihilating radiation produced by a tracer outside the body, and the collected information is processed by a computer to obtain a reconstructed image.

SPECT uses a radioactive isotope as a tracer, and the tracer is injected into the human body so that the tracer is concentrated on an organ to be examined, thus making the organ a source of γ-rays, and the distribution of radioactivity in organ tissue is recorded outside the body using detectors that rotate around the human body. One set of data is obtained when the detectors rotate to one angle, and several sets of data can be obtained when the detectors rotate a full circle. From said data, a series of tomographic planar images can be created, and a computer reconstructs the imaging in a cross-sectional manner.

PET and SPECT extend histopathological examination from a molecular level to display of local biochemistry of a tissue, and the provided images are images of human physiological metabolism, which are good at functional imaging and can detect functional and metabolic changes in the disease occurrence and development process; while CT and MRI are good at accurately reflecting morphological and structural changes. In existing methods, CT or MRI may be used for attenuation correction of PET or SPECT images. That is, PET or SPECT and CT or MRI are fused into one, so that functional and anatomical image information can complement one another to achieve better recognition and diagnosis.

The medical imaging system may include a separate computer device having the aforementioned medical imaging device and/or connected to the medical imaging device, and/or a computer device connected to an Internet cloud. The computer device may be connected by means of the Internet to the medical imaging device or a memory for storing medical images. An imaging method may be independently or jointly implemented by the aforementioned medical imaging device, the computer device connected to the medical imaging device, and the computer device connected to the Internet cloud.

In addition, a medical imaging workstation may be disposed locally at the medical imaging device. That is, the medical imaging workstation is disposed near to the medical imaging device, and the medical imaging workstation and medical imaging device may be located together in a scanning room, an imaging department, or a same medical institution. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SaaS)”. SaaS can exist between hospitals, between a hospital and an imaging center, or between a hospital and a third-party online diagnosis and treatment service provider.

In some embodiments, the medical imaging device may be used to perform scanning and imaging on a body part of the human body or other living entities. However, the present application is not limited thereto, and the medical imaging device may also be used to perform scanning and imaging on a non-living entity.

The following is a specific description of the embodiments of the present application with reference to the drawings.

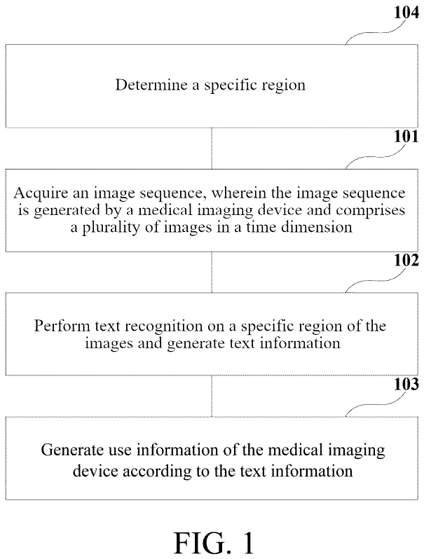

The embodiments of the present application provide a method for acquiring use information of a medical imaging device. FIG. 1 is a schematic diagram of the method for acquiring use information of a medical imaging device according to an embodiment of the present application. As shown in FIG. 1, the method includes:
step 101: acquiring an image sequence, wherein the image sequence is generated by the medical imaging device and includes a plurality of images in a time dimension;
step 102: performing text recognition on a specific region of the images and generating text information; and
step 103: generating use information of the medical imaging device according to the text information.

According to the above embodiment, text recognition is performed on a specific region of an image in an image sequence generated by a medical imaging device, and use information of the medical imaging device is generated according to the text information obtained by the recognition. As such, since the text recognition does not need to be performed within the entire range of the image and only a small amount of text recognition is required within the specific region of the image, the workload of text recognition can be reduced, computational resources can be saved, and the efficiency of generating the use information of the medical imaging device can be improved.

In some embodiments, in step 101, a plurality of images in a time dimension (i.e., an image sequence) are acquired from the medical imaging device, so that use information of the medical imaging device over a period of time can be acquired from information contained in the image sequence.

The use information may include identifier information, operating mode information, operating state information and the like of the medical imaging device. Alternatively, the use information may also be information further generated according to the above information, for example, the use information may also include information such as a usage rate, a serviceability rate, a failure rate, a functional development rate, a yield rate and the like of the medical imaging device.

The use information may be used as a basis for performance management of a medical imaging device. For example, according to the usage rates of medical imaging devices, the medical imaging devices can be appropriately allocated, and “unused”, “inefficiently running” medical imaging devices are quickly allocated to departments in need thereof for full use; alternatively, repair or maintenance can be quickly performed on the medical imaging devices according to information such as the usage rates or failure rates of the medical imaging devices; and so on and so forth.

In some embodiments, the image sequence may be an image sequence output from a video transmission interface of the medical imaging device. The image sequence may be an image sequence output in real time by the video transmission interface, so that images output by the medical imaging device over a full period of time can be acquired. Therefore, the accuracy and reliability of the generated use information of the medical imaging device can be ensured.

The video transmission interface of the medical imaging device may include various interfaces capable of outputting video data, e.g., an interface dedicated to video transmission such as an HDMI interface, a DP interface, a DVI interface, or a VGA interface, a network communication interface such as an RJ45 interface, or a data transmission interface such as a USB interface. The image sequence is acquired from an original video transmission interface of the medical imaging device, without the need to change the hardware structure of the medical imaging device, so that the invasiveness of the hardware connection of the medical imaging device is reduced. In addition, medical imaging devices are generally provided with video transmission interfaces, so the above-described method according to the embodiment of the present application has good applicability.

In some embodiments, as shown in FIG. 1, the method further includes:
step 104: determining a specific region.

Step 104 may be performed before step 101, or may be performed after step 101.

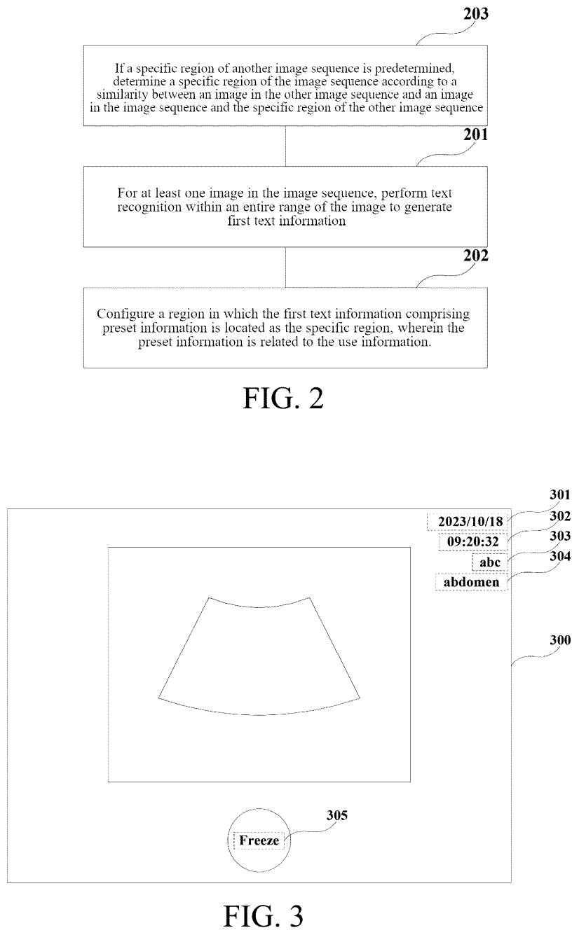

FIG. 2 is a schematic diagram of the implementation of step 104 according to an embodiment of the present application. In some embodiments, as shown in FIG. 2, step 104 may include:
step 201: for at least one image in the image sequence, performing text recognition within an entire range of the image to generate first text information; and
step 202: configuring a region in which the first text information including preset information is located as the specific region, wherein the preset information is related to the use information.

According to the above-described embodiment, the text recognition is performed within the entire range of the image, and the region including the preset information related to the use information is configured as the specific region. In this way, the specific region in the image can be automatically determined. Compared with the manner in which a text position is selected manually in the prior art, the operation steps can be simplified and the convenience can be improved. Furthermore, the above manner for determining the specific region is applicable to a plurality of medical systems or different versions of medical systems, thus, the applicability is good, and convenient maintenance can be achieved.

In some embodiments, the specific region is a partial region within the entire range of the image, the specific region being smaller than the size of the entire image.

In some embodiments, the determined specific region may be saved as a configuration text (e.g., in the form of JSON, YML, XML, etc.) for use at the next round of recognition.

In some embodiments, in step 201, a portion of the images may be selected from the image sequence for text recognition within the entire image range. The selected portion of the images may be a plurality of images in the image sequence, so that the reliability of the determined specific region can be improved. But the present application is not limited thereto, and this portion of the images may also be one image in the image sequence, so as to reduce the computation load for the specific region.

In some embodiments, scan images and related fields and the like may be included in the images. The related fields include, for example, date information, time information, patient information, and identifier information, operating mode information, operating state information and the like of the medical imaging device.

In step 201, when the text recognition is performed on the entire range of the image, one or more pieces of first text information may be generated according to the related fields within the entire image range, and each piece of first text information may correspond to a region and is located in said region.

The first text information may be generated by performing text recognition in various manners. For example, a conventional OCR (Optical Character Recognition) technique may be used to perform text recognition, including performing text region positioning, text image correction, row and column division, classifier recognition, post-processing, and the like on an input image; alternatively, a depth learning-based OCR technique may be used for text recognition, for example, text recognition based on a PaddleOCR frame.

The region corresponding to the first text information may be determined in various manners. For example, the region in which the first text information is located is determined based on a connected domain, an edge feature, a stroke feature, a texture feature, etc. For the specific manner of text recognition, reference may be made to the related art, and further description will not be made here.

In some embodiments, in step 202, when the region in which the first text information is located includes the preset information, the region in which the first text information is located may be configured as the specific region of the image.

The preset information is related to the use information of the medical imaging device. The preset information may include at least one piece of index information for generating the use information, and each piece of index information includes at least one piece of index data.

For example, the index information may include identifier information, operating mode information, operating state information and the like of the medical imaging device.

The identifier information of the medical imaging device includes, for example, type information and the like of the medical imaging device, for example, the model of an ultrasound probe and the like.

The operating mode information includes, for example, scan site information and the like, such as the abdomen, neck, etc, or the liver, gallbladder, kidney, breast, thyroid, etc.

The operating state information includes various information related to the operating state of the medical imaging device. Taking an ultrasound device as an example, the operating state of the ultrasound device may include the still state (outputting a still ultrasound image), non-still state (outputting a real-time ultrasound image), etc.

In some embodiments, in the preset information, one piece of index information may include all possible index data items which may cover different medical systems. As a result, the applicability of the method described in the embodiments of the present application can be further improved.

For example, taking an ultrasound device as an example, the index data indicating the abdomen in the operating mode information (index information) may include: abdomen, adult abdomen, Abdomen, etc.; the index data indicating the still state in the operating state information (index information) may include: still, freeze, etc.; and the index data indicating the non-still state may include non-still, unfreeze, live, etc.

In some embodiments, when the first text information is the same as the index data of the preset information, it is determined that the region in which the first text information is located includes the preset information, and the region in which the first text information is located is configured as the specific region.

For example, when the first text information is “adult abdomen”, which is the same as the index data “adult abdomen” of the preset information, the region in which the first text information is located is configured as the specific region.

In some embodiments, when a similarity between the first text information and the index data of the preset information is greater than a preset threshold, it can also be determined that the region in which the first text information is located includes the preset information, so that the region in which the first text is located is configured as the specific region. As a result, the method can be compatible with more medical systems or software versions, maintenance costs can be reduced, and the applicability of the method described in the embodiments of the present application can be further improved.

The similarity between the first text information and the index data may be determined in various manners, for example, the similarity may be computed based on character strings, a corpus, or the like, and for the specific computational manner, reference may be made to the related art, and further description will not be made here. The preset threshold may be an empirical value or a value determined according to the actual situation.

For example, the first text information is “the abdomen of an adult” and the similarity between the first text information and the index data “adult abdomen” exceeds 98%; in such a case, the region in which the first text information is located may also be configured as the specific region.

In some embodiments, in the preset information, there may be a plurality of index information used to generate the use information. In this case, a specific region corresponding to each piece of index information may be specified. For example, if the first text information is the same as first index data of first index information among the plurality of pieces of index information or a similarity between the first text information and the first index data is greater than a preset threshold, the region in which the first text information is located is configured as the specific region of the first index information.

For example, the preset information may include three pieces of index information, i.e., identifier information, operating mode information, and operating state information of the medical imaging device. A corresponding specific region may be determined for each of the three pieces of index information.

In some embodiments, the preset information may be prestored in a database, so as to facilitate a query or search. For example, after the first text information is acquired, the database can be queried to determine whether index data that is the same as the first text information or has a similarity exceeding a preset threshold exists therein, and if the index data exists in the database, the region in which the first text information is located can be configured as the corresponding specific region.

The database may be of various types. For example, a non-relational database (e.g., Elasticsearch, MongoDB, etc.) may be used, in which preset information (e.g., an index dataset storing three pieces of index information, i.e., identifier information, operating mode information, and operating state information of the medical imaging device) is stored in a segmented form, and is retrieved using an indexed query.

For another example, an in-memory database (e.g., Redis, Memecache, etc.) may be used, in which preset information is stored in a hash table, and is retrieved using a keyword query; alternatively, a Bloom filter is used for storage, and comparison is made by determining whether each keyword is present in the preset information (for example, an index dataset including three pieces of index information, i.e., identifier information, operating mode information, and operating state information of the medical imaging device).

FIG. 3 is a schematic diagram of a specific region according to an embodiment of the present application. FIG. 3 shows an example of a specific region in an ultrasound image, and it can be understood that the following description of the specific region is also applicable to other types of images.

As shown in FIG. 3, the image 300 is an image in an image sequence. Text recognition is performed on an entire image range of the image 300 to obtain a plurality of pieces of first text information, e.g., “2023/10/18”, “09:20:32”, “abc”, “abdomen”, “freeze”, and regions 301 to 305 corresponding to the plurality of pieces of first text information.

Each piece of first text information is used as a keyword to search a database having the preset information stored therein, the database including, for example, an identifier information database, an operating mode information database, and an operating state information database.

When the identifier information database includes the first text information “abc”, a region 303 in which the first text information “abc” is located is used as a specific region of the identifier information; when the operating mode information database includes the first text information “abdomen”, a region 304 in which the first text information “abdomen” is located is used as a specific region of the operating mode information; and when the operating state information database includes the first text information “freeze”, a region 305 in which the first text information “freeze” is located is used as a specific region of the operating state information.

In some embodiments, when more than one image is selected from the image sequence to perform text recognition on the entire range to determine a specific region, candidate specific regions may be generated respectively from the images and a final specific region may be determined from the plurality of candidate specific regions. For the manner of determining the candidate specific regions, reference may be made to the above disclosure, and further description will not be made here.

In some embodiments, the final specific region may be a region selected from the plurality of candidate specific regions; for example, a region in which the first text information having the highest similarity is located is selected as the specific region from the plurality of candidate specific regions according to the similarity between the first text information and the index data. But the present application is not limited thereto, and the specific region may be selected according to other criteria.

Alternatively, the final specific region may be an average value of the plurality of candidate specific regions. For example, the specific region may be represented by position information which may include at least one of: coordinate information (x, y) of a first pixel in the region in which the first text information is located, height information (h) of the region in which the first text information is located, or width information (w) of the region in which the first text information is located. Correspondingly, the candidate specific regions may also be represented by the above position information.

The above x, y, h, and w may be represented by pixel positions in the image. For example, the image includes 1920*1080 pixels, and the position information (x, y, h, w) may be represented as [1795, 45, 125, 25].

In the position information of the specific region, the position information of the first pixel may be an average value of the position information of the first pixel in the candidate specific regions. The height information may be an average value of the height information of the candidate specific regions. The width information may be an average value of the width information of the candidate specific regions.

In some embodiments, the first pixel may be any pixel within the region in which the first text information is located. For example, when the region in which the first text information is located is a rectangle, the first pixel may be a pixel corresponding to the upper left corner of the rectangle; alternatively, the first pixel may be a pixel corresponding to the center of the rectangle, and so on.

In some embodiments, as shown in FIG. 2, step 104 may further include:
step 203: if a specific region of another image sequence is predetermined, determining a specific region of the image sequence according to a similarity between an image in the other image sequence and an image in the image sequence and the specific region of the other image sequence.

For example, if the similarity between an image in the other image sequence and an image in the image sequence is greater than a first threshold, the specific region of the other image sequence is used as the specific region of the image sequence.

The similarity between images may be expressed in various manners, e.g., a similarity between image data, or a similarity between digital fingerprints (or digital digests) of images, or the like. For the specific manner of obtaining the similarity between images, reference may be made to the related art, and further description will not be made here.

For example, for the same model and the same (medical system) software version, when the similarity between images is 98% or more, the original configuration file (specific region) may be reused.

According to the above-described embodiment, text recognition may be performed on the entire image range to generate the first text information and corresponding region information. After the database is searched for each index information in the index database, the position information of the specific region can be determined and written into the configuration file for storage. The configuration file may be used as the current independent configuration file of the current medical imaging device for subsequent text recognition operations; alternatively, when the similarity between images is greater than the first threshold, the configuration file may also be used as a configuration file for other medical imaging devices, saving computational resources while saving a significant amount of human cost resources.

In some embodiments, in step 102, when the text recognition is performed on the specific region of the images to generate the text information, the result of the text recognition may be converted with reference to the preset information or the database including the preset information to generate standardized text information.

For example, in the operating mode information (index information) database, the dataset (index data) representing the abdomen includes abdomen, adult abdomen, and Abdomen, and if the result of the text recognition for the specific region is Abdomen, that is, the result of the text recognition is the same as at least one item in the dataset representing the abdomen, then the result of the text recognition may be normalized as “abdomen”; alternatively, if the result of the text recognition is “the abdomen of an adult”, that is, the similarity between the result of the text recognition and at least one item in the dataset representing the abdomen exceeds a preset threshold, the result of the text recognition may also be normalized as “abdomen”.

In some embodiments, in step 103, the use information of the medical imaging device is generated according to the text information. Taking the use information including identifier information, operating mode information, and operating state information of the medical imaging system as an example, when any one of the above three changes, the information of the above three and time information are recorded. For example, the use information may include a plurality of entries, and when any one of the identifier information, the operating mode information, and the operating state information changes, a new entry is created, and the start time and the end time can be recorded in each entry.

Table 1 is an example of the use information. As shown in Table 1, Entry 1 indicates that an ultrasound probe ABC scans an adult abdomen during the period from 9:00 to 9:30 to output a real-time scan image; and Entry 2 indicates that an ultrasound probe ABC scans an adult abdomen during the period from 19:10 to 19:15 to output a still scan image.

TABLE 1
Examples of use information

| | | | | | |
|---|---|---|---|---|---|
| Entry 1 | 9:00 | 9:30 | ABC | Adult abdomen | FREEZE |
| Entry 2 | 19:10 | 19:15 | ABC | Adult abdomen | Live |

The present application is not limited thereto, and the use information may also include other content or be in other forms.

In some embodiments, the use information may be written into the database for subsequent performance analysis or management of the medical imaging device based on the use information.

It should be noted that FIGS. 1-3 merely schematically illustrate the embodiments of the present application, but the present application is not limited thereto. For example, the order of execution between the operations may be appropriately adjusted. In addition, some other operations may be added or some operations may be omitted. Those skilled in the art could make appropriate variations according to the above disclosure, rather than being limited by the disclosures of FIGS. 1-3.

As can be seen from the above embodiments, text recognition is performed on a specific region of an image in an image sequence generated by a medical imaging device, and use information of the medical imaging device is generated according to text information obtained by the recognition. As such, since text recognition does not need to be performed within the entire range of the image, the workload of text recognition can be reduced, computational resources can be saved, and the efficiency of generating the use information of the medical imaging device can be improved.

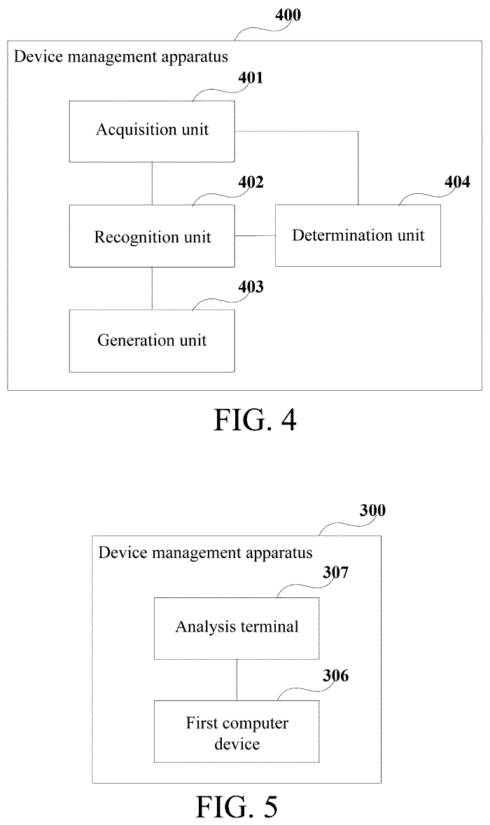

The embodiments of the present application further provide an imaging device management apparatus, and repetitive content from the aforementioned embodiments is not repeated herein. FIG. 4 is a schematic diagram of an imaging device management apparatus according to an embodiment of the present application. As shown in FIG. 4, the imaging device management apparatus 400 includes: an acquisition unit 401, a recognition unit 402, and a generation unit 403.

The acquisition unit 401 acquires an image sequence, where the image sequence is generated by a medical imaging device and includes a plurality of images in a time dimension; the recognition unit 402 performs text recognition on a specific region of the images and generates text information; and the generation unit 403 generates use information of the medical imaging device according to the text information.

In some embodiments, as shown in FIG. 4, the apparatus 400 further includes: a determination unit 404.

The determination unit 404 determines the specific region. For at least one image in the image sequence, the determination unit performs text recognition within an entire range of the image to generate first text information, and configures a region in which the first text information including preset information is located as the specific region, where the preset information is related to the use information.

In some embodiments, the preset information includes at least one piece of index information for generating the use information, and each piece of index information includes at least one piece of index data.

In some embodiments, when the first text information is the same as the index data or a similarity between the first text information and the index data is greater than a preset threshold, the region in which the first text information is located includes the preset information.

In some embodiments, there are a plurality of pieces of index information, and when the first text information is the same as first index data of first index information among the plurality of pieces of index information or a similarity between the first text information and the first index data is greater than a preset threshold, the region in which the first text information is located is configured as a specific region of the first index information.

In some embodiments, if a specific region of another image sequence is predetermined, the determination unit 404 determines a specific region of the image sequence according to a similarity between an image in the other image sequence and an image in the image sequence and the specific region of the other image sequence.

In some embodiments, if the similarity between the image in the other image sequence and the image in the image sequence is greater than a first threshold, the specific region of the other image sequence is used as the specific region of the image sequence.

In some embodiments, the image sequence is an image sequence output by a video transmission interface of the medical imaging device.

In some embodiments, the preset information is prestored in a database.

In some embodiments, the specific region is represented by position information, the position information including at least one of: coordinate information of a first pixel in the region in which the first text information is located, height information of the region in which the first text information is located, or width information of the region in which the first text information is located.

FIG. 5 is another schematic diagram of the device management apparatus 300 according to an embodiment of the present application. As shown in FIG. 5, the device management apparatus 300 includes an analysis terminal 307 and a first computer device 306. The analysis terminal 307 may be directly connected to a video transmission interface of a medical imaging device, so as to receive an image sequence output by the medical imaging device from the video transmission interface.

In some embodiments, the analysis terminal 307 may be a microprocessor. For example, the analysis terminal 307 may include a processor and a memory, and may run a system such as embedded (Linux/Debian) system. The memory may store programs therein that may be processed using a script language (shell/python), image processing techniques (ffmpeg, opencv), OCR text recognition, and the like.

The analysis terminal 307 may be configured to implement the function of at least one of the acquisition unit 401, the recognition unit 402, and the generation unit 403 of the imaging device management apparatus 300 illustrated in FIG. 4. For the specific implementation, reference may be made to the aforementioned embodiments, and the details are not described herein again. In addition, the analysis terminal 307 may also perform preprocessing, such as data cleansing, binarization, and grayscale processing, on the images in the image sequence.

In some embodiments, the first computer device 306 may be a computer server or a cloud platform or a workstation, etc., and the embodiments of the present application are not limited thereto.

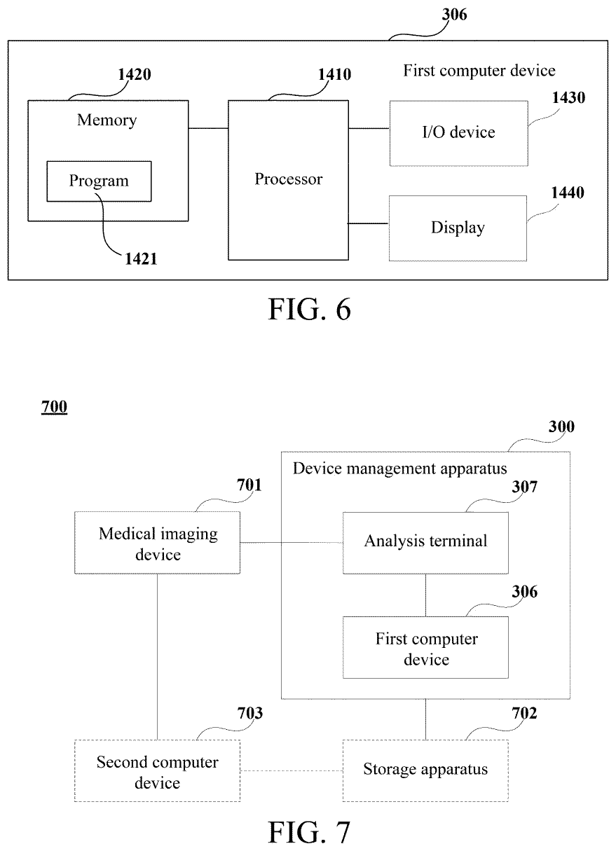

FIG. 6 is a schematic diagram of the first computer device 306 according to the present application. As shown in FIG. 6, the first computer device 306 may include: one or more processors (e.g., central processing units (CPU)) 1410 and one or more memories 1420; the memory 1420 being coupled to the processor 1410. The memory 1420 may store a program 1421 and/or data. The program 1421 is executed under the control of the processor 1410. The memory 1420 may include, for example, a ROM, a floppy disk, a hard disk, an optical disk, a magneto-optical disk, a CD-ROM, or a non-volatile memory card.

In some embodiments, some or all of the functions of the imaging device management apparatus 300 may be integrated into the processor 1410 for implementation. The processor 1410 is configured to implement the function of at least one of the recognition unit 402, the generation unit 403, and the determination unit 404 of the imaging device management apparatus 300 illustrated in FIG. 4. For the implementation of the processor 1410, reference can be made to the foregoing embodiments, and the details are not described herein again.

As shown in FIG. 6, the first computer device 306 may further include: an input device 1430 and a display 1440. The functions of the foregoing components are similar to those in the prior art, and details are not described herein again. It should be noted that the first computer device 306 does not necessarily include all of the components shown in FIG. 6. In addition, the first computer device 306 may further include components not shown in FIG. 6, for which reference may be made to the related art.

As can be seen from the above embodiments, text recognition is performed on a specific region of an image in an image sequence generated by a medical imaging device, and use information of the medical imaging device is generated according to text information obtained by the recognition. As such, since text recognition does not need to be performed within the entire range of the image, the workload of text recognition can be reduced, computational resources can be saved, and the efficiency of generating the use information of the medical imaging device can be improved.

The embodiments of the present application further provide a medical imaging system. FIG. 7 is a schematic diagram of a medical imaging system according to an embodiment of the present application. As shown in FIG. 7, the medical imaging system 700 includes a medical imaging device 701 and a device management apparatus 300. For the related features of the medical imaging device 701 and the device management apparatus 300, reference may be made to the foregoing embodiments, and the repetitive content is not repeated herein.

The medical imaging device 701 generates an image sequence, where the image sequence includes a plurality of images in a time dimension; and the device management apparatus 300 acquires the image sequence, performs text recognition on a specific region of the images of the image sequence and generates text information, and generates use information of the medical imaging device according to the text information.

In some embodiments, the image processing device 300 is connected to a video transmission interface of the medical imaging device 701 to acquire the image sequence from the video transmission interface.

In some embodiments, as shown in FIG. 7, the medical imaging system 700 may further include a second computer device 703 connected to the medical imaging device 701. The second computer device 703 may run a medical system (e.g., PACS/RIS, etc.) for managing the medical imaging device 701. The structure of the second computer device 703 may be similar to the structure of the first computer device 306.

In some embodiments, the first computer device 306 and the second computer device 703 may be provided separately or may be integrated together.

In some embodiments, as shown in FIG. 7, the medical imaging system 700 may further include a storage apparatus 702 that may be configured to store at least one of the preset information, the configuration text, and the use information in the foregoing embodiments. The storage apparatus 702 may be provided separately, or may be integrated in the first computer device 306 or the second computer device 703.

The embodiments of the present application further provide a computer-readable program. When executed, the program causes a computer to perform the method for acquiring use information of a medical imaging device described in the aforementioned embodiments in the apparatus, system or computer device.

The embodiments of the present application further provide a storage medium storing a computer-readable program. The computer-readable program causes a computer to perform the method for acquiring use information of a medical imaging device described in the aforementioned embodiments in the apparatus, system or computer device.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. One or a plurality of the functional blocks and/or one or a plurality of combinations of the functional blocks described relative to the figures may also be implemented as a combination of computing devices, such as a combination of a DSP and a microprocessor, a plurality of microprocessors, one or a plurality of microprocessors in communication combination with a DSP, or any other such configuration. The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A method for acquiring use information of a medical imaging device, characterized by comprising:
acquiring an image sequence, wherein the image sequence is generated by the medical imaging device and comprises a plurality of images in a time dimension;
performing text recognition on a specific region of the images and generating text information; and
generating use information of the medical imaging device according to the text information.

2. The method according to claim 1, wherein
the image sequence is an image sequence output by a video transmission interface of the medical imaging device.

3. The method according to claim 1, further comprising: determining the specific region, wherein the determining the specific region comprises:
for at least one image in the image sequence, performing text recognition within an entire range of the image to generate first text information; and
configuring a region in which the first text information comprising preset information is located as the specific region, wherein the preset information is related to the use information.

4. The method according to claim 3, wherein
the preset information is prestored in a database.

5. The method according to claim 3, wherein
the preset information comprises at least one piece of index information for generating the use information, and each piece of index information comprises at least one piece of index data.

6. The method according to claim 5, wherein
when the first text information is the same as the index data or a similarity between the first text information and the index data is greater than a preset threshold, the region in which the first text information is located comprises the preset information.

7. The method according to claim 5, wherein
there are a plurality of pieces of index information, and when the first text information is the same as first index data of first index information among the plurality of pieces of index information or a similarity between the first text information and the first index data is greater than a preset threshold, the region in which the first text information is located is configured as a specific region of the first index information.

8. The method according to claim 7, wherein
the specific region is represented by position information, the position information comprising at least one of: coordinate information of a first pixel in the region in which the first text information is located, height information of the region in which the first text information is located, or width information of the region in which the first text information is located.

9. The method according to claim 3, wherein the determining the specific region further comprises:
if a specific region of another image sequence is predetermined, determining a specific region of the image sequence according to a similarity between an image in the other image sequence and an image in the image sequence and the specific region of the other image sequence.

10. The method according to claim 9, wherein
if the similarity between the image in the other image sequence and the image in the image sequence is greater than a first threshold, the specific region of the other image sequence is used as the specific region of the image sequence.

11. An imaging device management apparatus, characterized by comprising:
an acquisition unit configured to acquire an image sequence, wherein the image sequence is generated by a medical imaging device and comprises a plurality of images in a time dimension;
a recognition unit configured to perform text recognition on a specific region of the images and generate text information; and
a generation unit configured to generate use information of the medical imaging device according to the text information.

12. The apparatus according to claim 11, further comprising:
a determination unit configured to determine the specific region, wherein for at least one image in the image sequence, the determination unit performs text recognition within an entire range of the image to generate first text information, and configures a region in which the first text information comprising preset information is located as the specific region, wherein the preset information is related to the use information.

13. The apparatus according to claim 12, wherein
the preset information comprises at least one piece of index information for generating the use information, and each piece of index information comprises at least one piece of index data.

14. The apparatus according to claim 13, wherein
when the first text information is the same as the index data or a similarity between the first text information and the index data is greater than a preset threshold, the region in which the first text information is located comprises the preset information.

15. The apparatus according to claim 13, wherein
there are a plurality of pieces of index information, and when the first text information is the same as first index data of first index information among the plurality of pieces of index information or a similarity between the first text information and the first index data is greater than a preset threshold, the region in which the first text information is located is configured as a specific region of the first index information.

16. The apparatus according to claim 12, wherein
if a specific region of another image sequence is predetermined, the determination unit determines a specific region of the image sequence according to a similarity between an image in the other image sequence and an image in the image sequence and the specific region of the other image sequence.

17. The apparatus according to claim 16, wherein
if the similarity between the image in the other image sequence and the image in the image sequence is greater than a first threshold, the specific region of the other image sequence is used as the specific region of the image sequence.

18. A medical imaging system, characterized by comprising:
a medical imaging device configured to generate an image sequence, wherein the image sequence comprises a plurality of images in a time dimension; and
a device management apparatus configured to acquire the image sequence, perform text recognition on a specific region of the images of the image sequence and generate text information, and generate use information of the medical imaging device according to the text information.

19. The system according to claim 18, wherein
the image processing device is connected to a video transmission interface of the medical imaging device to acquire the image sequence from the video transmission interface.

20. The system of claim 18, further comprising: determining the specific region, wherein the determining the specific region comprises:
for at least one image in the image sequence, performing text recognition within an entire range of the image to generate first text information; and
configuring a region in which the first text information comprising preset information is located as the specific region, wherein the preset information is related to the use information.
